(19) United States
(12) Patent Application Publication
(10) Pub. No.: US 2004/0235968 A1
(43) Pub. Date: Nov. 25, 2004

(54) Process and apparatus for controlling flow in a multiphase reactor

(75) Inventors: Yi Jiang, Ponca City, OK (US); Jianping Zhang, Ponca City, OK (US); Rafael L. Espinoza, Ponca City, OK (US)

Correspondence Address: DAVID W. WESTPHAL, CONOCOPHILLIPS COMPANY - I.P. Legal, P.O. BOX 1267, PONONCA CITY, OK, 74602-1267, US

(73) Assignee: ConocoPhillips Company, Houston, TX

(21) Appl. No.: 10/807,851

(22) Filed: Mar. 24, 2004

Related U.S. Application Data
(60) Provisional application No. 60/458,818, filed on Mar. 28, 2003.

(51) Int. Cl. B01J 8/04; B01J 10/00; C07C 27/26

(52) U.S. Cl. 518/726; 422/188; 422/190; 422/191

(57) ABSTRACT

The present invention provides an apparatus and method for controlling the hydrodynamics within a gas agitated multiphase reactor at a given gas linear velocity. The embodiments of the present invention involve novel configurations of the multiphase reactor internal structures. In general, the configurations comprise a plurality of discrete reaction flow zones created by arranging the internal structures of a multiphase reactor.

### CROSS-REFERENCE TO RELATED APPLICATIONS

This application claims benefit of U.S. provisional application Ser. No. 60/458,818, filed Mar. 28, 2003, and is related to commonly assigned, co-pending U.S. utility application Ser. No. 10/402,498, filed Mar. 28, 2003, and entitled “Gas Agitated Multiphase Catalytic Reactor with Reduced Backmixing,” both which are hereby incorporated herein by reference.

### STATEMENT REGARDING FEDERALLY SPONSORED RESEARCH OR DEVELOPMENT

Not applicable.

### FIELD OF THE INVENTION

The present invention relates to an apparatus and method for controlling the flow hydrodynamics within a gas agitated multiphase reactor. In particular, the present invention provides a new and improved method for producing -hydrocarbons from synthesis gas.

### BACKGROUND OF THE INVENTION

Natural gas, found in deposits in the earth, is an abundant energy resource. For example, natural gas commonly serves as a fuel for heating, cooking, and power generation, among other things. The process of obtaining natural gas from an earth formation typically includes drilling a well into the formation. Wells that provide natural gas are often remote from locations with a demand for the consumption of the natural gas.

Thus, natural gas is conventionally transported large distances from the wellhead to commercial destinations in pipelines. This transportation presents technological challenges due in part to the large volume occupied by a gas. Because the volume of a gas is so much greater than the volume of a liquid containing the same number of gas molecules, the process of transporting natural gas typically includes chilling and/or pressurizing the natural gas in order to liquefy it. However, this contributes to the final cost of the natural gas. Further, naturally occurring sources of crude oli used for liquid fuels such as gasoline and middle distillates have been decreasing and supplies are not expected to meet demand in the coming years. Middle distillates typically include heating oil, jet fuel, diesel fuel, and kerosene. Fuels that are liquid under standard atmospheric conditions have the advantage that in addition to their value, they can be transported more easily in a pipeline than natural gas.

Thus, for all of the above-described reasons, there has been interest in developing technologies for converting natural gas to more readily transportable liquid fuels, i.e. to fuels that are liquid at standard temperatures and pressures. One method for converting natural gas to liquid fuels involves two sequential chemical transformations. In the first transformation, natural gas or methane, the major chemical component of natural gas, is reacted with oxygen to form syngas, which is a combination of carbon monoxide gas and hydrogen gas. In the second transformation, known as the Fischer-Tropsch process, carbon monoxide is reacted with hydrogen to form organic molecules containing carbon and hydrogen. Those organic molecules containing only carbon and hydrogen are known as hydrocarbons. In addition, other organic molecules containing oxygen in addition to carbon and hydrogen known as oxygenates may be formed during the Fischer-Tropsch process. Hydrocarbons having carbons linked in a straight chain are known as aliphatic hydrocarbons that may include paraffins and/or olefins. Paraffins are particularly desirable as the basis of synthetic diesel fuel.

Typically the Fischer-Tropsch product stream contains hydrocarbons having a range of numbers of carbon atoms, and thus having a range of molecular weights. Thus, the Fischer-Tropsch products produced by conversion of natural gas commonly contains a range of hydrocarbons including gases, liquids and waxes. Depending on the molecular weight product distribution, different Fischer-Tropsch product mixtures are ideally suited to different uses. For example, Fischer-Tropsch product mixtures containing liquids may be processed to yield gasoline, as well as heavier middle distillates. Hydrocarbon waxes may be subjected to additional processing steps for conversion to liquid and/or gaseous hydrocarbons. Thus, in the production of a Fischer-Tropsch product stream for processing to a fuel it is desirable obtain primarily hydrocarbons that are liquids and waxes (e.g. C₅₊ hydrocarbons).

Fischer-Tropsch reactions are generally carried out in gas agitated multiphase reactors. In a gas agitated multiphase (i.e., gas/liquid/solid) reactor, a gas feedstream comprising a reactant gas and optionally an inert gas, provides the required stirring action to drive liquid and solid flow. It offers an attractive way to carry out gas-liquid and gas-liquid-solid reactions due to its simple construction and operation. In such reactors, local flow structure, turbulence, gas holdup distribution, and solid phase distribution if present are interrelated in a complex way with the operating and design variables. Fluid dynamics of the reactors may change considerably with variations in physicochemical properties and the scale of the reactor. This complexity of fluid dynamics can cause difficulty in efficient design and scale-up of gas agitated multiphase reactors.

One key consideration for the design of industrial scale gas agitated multiphase reactors is the hydrodynamics data from bench-scale and pilot scale experiments. However, extrapolation from small scale to large-scale reactors is quite difficult and can lead to flawed results. Most hydrodynamics experiments of gas agitated multiphase reactors are conducted using reactors having a diameter of less than one meter, often only a few inches. Therefore there is a need for reactor designs that can be more readily scaled up, particularly for high pressure, and/or high temperature applications.

Moreover, gas agitated multiphase reactors, including gas-solid, gas-liquid, and gas-liquid-solid reactors which are widely used in chemical, petrochemical and biochemical processes often suffer from a high degree of backmixing. During backmixing, gas introduced into a reactor will tend to have a higher gas holdup and upward flow in the central region of the reactor while there is a lower gas holdup and a downward flow in the annular or outer region of the reactor. Backmixing can affect many of the essential characteristics of the reactor and reaction including but not limited to conversion rates, productivity, mass transport capabilities, gas distribution and heat control. In addition, the backmixing phenomenon increases with increasing diameter of the reactor, which adds to the complexity of scale up from bench or pilot scale reactors to commercial size reactors.

Hence, there remains a need for reactor designs that are more efficient, flexible, cost effective, and/or productive for obtaining desirable hydrocarbon products. It is also highly desirable to find scaleup methods for gas agitated multiphase reactor designs that reduce the problems associated with scale up. The present invention is an improvement toward fulfilling one or more those needs.

### SUMMARY OF THE INVENTION

The present invention provides an apparatus and method for controlling the hydrodynamics within a gas agitated multiphase reactor, particularly for industrial scale reactors. The embodiments of the present invention involve novel configurations of the multiphase reactor internal structures. These configurations result in marked improvement in many areas with respect to multiphase reactors including but not limited to reduced backmixing, controllable axial dispersion coefficient, improved mass transport capabilities, smaller gas bubbles, improved reaction distribution, improved control over the heat of reaction and/or increased flexibility in reactor design. Any one or more of these benefits may result in decreased costs, increased conversion and increased productivity.

In accordance with the broad aspects of the present invention, the reactor internals are configured such that the whole flow domain behaves more like several discrete zones. The hydrodynamics in each zone approaches the hydrodynamics in a column with an equivalent diameter of the zone. The present invention contemplates one or more reaction regions within the reactor having one or more configurations of internal structures, wherein each reaction zone is in fluid communication with at least one adjacent reaction zone. The internal structures are preferably internal exchanger tubes (i.e., cooling or heating tubes) but can be additional active or non-active structures located within the reactor. The present invention is an improvement for all multiphase reactors with backmixing or gas distribution problems.

Accordingly, another embodiment of the present invention comprises an improved method for using a multiphase reactor with the configurations described and claimed herein. However, the present invention is preferably useful in Fischer-Tropsch multiphase reactors. Thus, another embodiment of the present invention comprises an improved method for producing hydrocarbons using a multiphase reactor with the configurations described and claimed herein.

These and other embodiments, features and advantages of the present invention will become apparent with reference to the following detailed description and drawings.

### BRIEF DESCRIPTION OF THE DRAWINGS

For a more detailed understanding of the present invention, reference is made to the accompanying Figures, wherein:

FIG. 1 shows an example of a gas agitated multiphase reactor with internal structures;

FIGS. 2a and 2b show time-averaged velocity profiles for a typical multiphase reactor without and with internal structures respectively;

FIG. 3 shows the liquid axial dispersion coefficient and productivity of a Fischer-Tropsch gas agitated multiphase reactor with a cobalt-based catalyst at various column diameters with different gas superficial velocities;

FIG. 4 shows a cross section of a multiphase reactor with circular zones;

FIG. 5 shows a cross section of a multiphase reactor with a diamond shaped zones;

FIG. 6 shows a cross section of a multiphase reactor with rectangular zones;

FIG. 7 shows a cross section of a multiphase reactor with parallel zones;

FIG. 8 shows a cross section of a multiphase reactor with internal tubular structures with walls permeable to gas/liquid and with solid phase retained outside said walls;

FIG. 9 shows a cross section of a multiphase reactor with internal tubular structures with walls permeable to gas/liquid and with solid phase retained inside said walls; and

FIG. 10 shows a cross section of a multiphase reactor with internal flat structures with walls permeable to gas/liquid, said structures arranged in parallel.

### DETAILED DESCRIPTION OF THE PREFERRED EMBODIMENTS

There are shown in the Figures, and herein will be described in detail, specific embodiments of the present invention with the understanding that the present disclosure is to be considered an exemplification of the principles of the invention, and is not intended to limit the invention to that illustrated and described herein. The present invention is susceptible to embodiments of different forms or order and should not be interpreted to be limited to the particular methods or compositions contained herein. In particular, various embodiments of the present invention provide a number of different configurations of the overall gas to liquid conversion process.

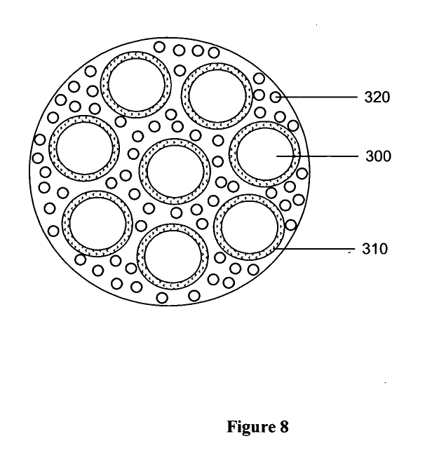

The present invention is directed toward improving the design and operation of a multiphase reactor. Depending upon the reaction(s) taking place, the configurations within the reaction zone of a multiphase reactor according to the present invention may result in a variety of benefits including but not limited to lower risk of scale up, optimized backmixing, improved gas distribution, improved mass transport capabilities, smaller gas bubbles, improved reaction distribution, improved control over the heat of reaction and/or increased flexibility and increased confidence in reactor design. When the permeable zone walls as shown in FIGS. 8, 9 and 10 are employed, the present invention provides an efficient way to withdraw the liquid products on-line and to perform the separation of liquid and solid catalysts without additional separation equipment. In general, embodiments of the present invention comprise arranging the internal structures of a multiphase reactor such that the whole flow domain is altered to behave more like multiple discrete small zones rather than a single large column reaction zone.

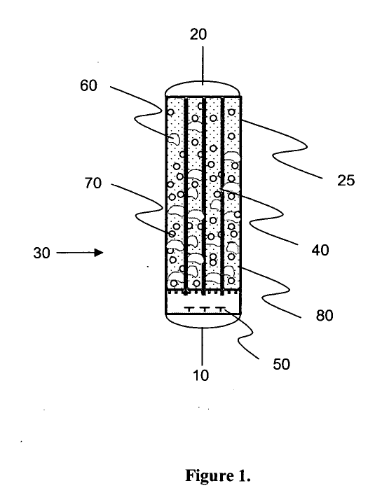

FIG. 1 shows a typical schematic of a gas agitated three-phase reactor 30 with internal cooling or heating tubes 40 and having bottom 10, top 20 and side walls 25. A gas phase is introduced to reactor 30 through bottom 10 and gas distributor 50 and exits through top 20. The rising gas acts to mix or agitate the slurry comprised of liquid and solid particles 80 inside reactor 30 as it moves upward during operation.

Such a gas agitated three-phase reactor can be either operated in the homogeneous flow regime or in the heterogeneous (chum-turbulent) flow regime mainly depending on the gas superficial velocity (U_{g}). In the homogeneous flow regime, small bubbles of gas (1-10 mm) are uniformly distributed in the slurry phase (liquid+solid catalyst particles). In the heterogeneous regime, however, small bubbles 70 combine in cluster to form larger bubbles 60 through bubble coalescences. Large bubbles 60 travel up through the reactor 30 at relatively high velocities (in the range of up to 1-2 m/s), and break up into small bubbles 70 through bubble breakages. Small bubbles 70 that co-exist with large bubbles 60 in the churn-turbulent regime are “entrained” in the liquid or slurry phase. Most of the commercial scale gas agitated multiphase reactors are operated in the churn-turbulent flow regime under elevated pressure because of the high interaction between bubbles and the high mass transfer rate between bubble and liquid phase, which are beneficial for achieving good reactor performance.

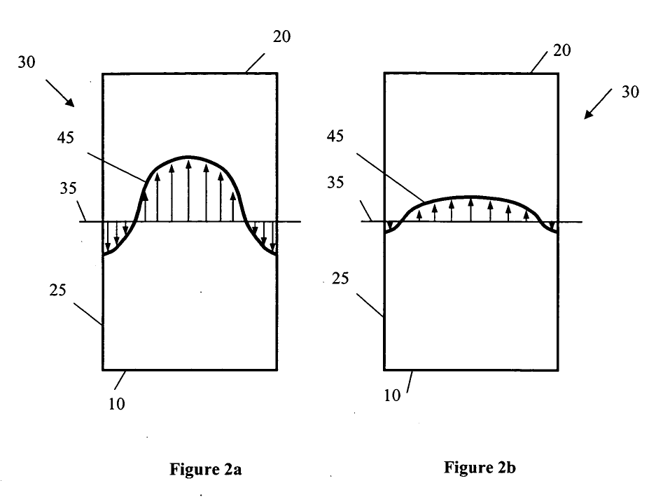

FIGS. 2a and 2b show a cross section of reactor 30 of FIG. 1. FIGS. 2a and 2b both show the tendency for the gas and slurry to have a greater upward flow in the central region and a greater downward flow at the wall regions 25 of the reactor. FIGS. 2a and 2b show a velocity profile, represented by lines 45, in a gas agitated reactor respectively with and without internal structures. Line 35 represents a baseline value of zero or no net flow in any direction. The area above line 35 represents a positive or upward flow and below line 35 represents negative or downward flow. As shown, the positive area under the curve is greater in the central region of the reactor and the negative area is greater near the walls 25 of reactor 30. The internal structures tend to flatten the velocity profile (FIG. 2b) by reducing the degree of backmixing. It should be appreciated that FIGS. 2a and 2b are not intended to limit the present invention to the particular reactor or to the exact flow distribution shown. FIGS. 2a and 2b are merely illustrative of the effect of internal structures on the overall velocity profile of a gas agitated multiphase reactor.

This phenomenon of liquid backmixing can be the result of many factors. For example, the degree of backmixing may be dependent upon the diameter of the reactor and the velocity of the gas phase. High gas velocity and large reactor diameter give a high degree of backmixing. For example, when a reactor of large diameter, e.g., about 0.6 to about 10 m, is operated in the churn-turbulent flow regime with a high gas velocity, e.g., about 12 to about 50 cm/s, the reactor will experience the motion of large gas bubbles (a cluster of large bubbles as shown in FIG. 1). However, using the principles of the present invention allows reactors with higher diameters and/or velocities to operate as though they were comprised of smaller diameter and/or velocities. Thus, one embodiment of the present invention is for a multiphase reactor comprising a reaction zone of a large diameter, preferably equal to or greater than 0.6 m, that behaves in a manner consistent with a smaller diameter reaction zone, i.e., smaller than the reactor diameter being used. The ratio of reactor height over reactor diameter is preferably between about 0.5 and about 20. The diameter of the smaller reaction regions is preferably between about 6 inches and about 24 inches (0.15 m-0.6 m). Each of the reaction zones preferably has a height to diameter ratio between about 7 and about 180.

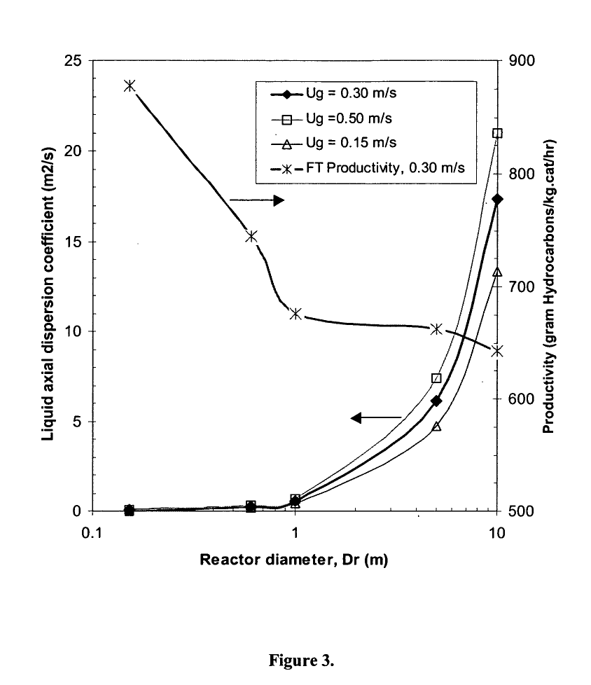

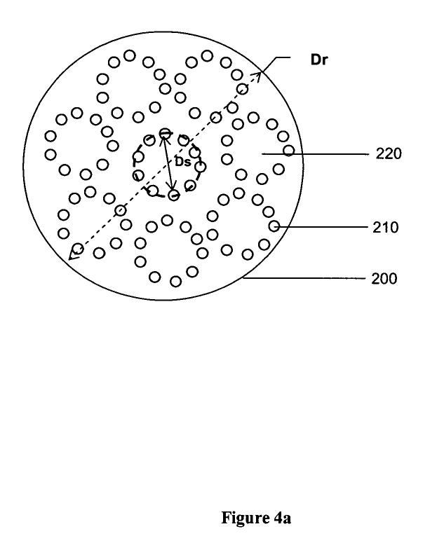

FIG. 3 which shows the liquid axial dispersion coefficient versus the reactor diameter is partly derived from the disclosure of Krishna et al. in ‘Liquid Phase Dispersion in Bubble Columns Operating in the Chum-turbulent Flow Regime’, Chem. Eng. Journal, volume 78, pages 43-51 (2000). FIG. 3 shows that the liquid axial dispersion coefficient is largely dependent on the reactor column diameter, i.e., it increases with increasing reactor diameter for a given reactor length, and it gradually decreases as the gas superficial velocity is decreased. For example, when using a small reactor diameter of 0.15 meters (6 inches) the liquid axial dispersion coefficient may be from about 0.017 m²/s to about 0.04 m²/s with gas linear velocities from about 0.05 m/s to about 0.6 m/s. For a larger more industrial scale reactors with a diameter of 5 meters (about 200 inches), the liquid axial dispersion coefficient may be about 3 m²/s to about 8 m²/s with gas linear velocities from about 0.05 to about 0.6 m/s, representing a 20 fold greater liquid axial dispersion coefficient than that of the 0.15-meter diameter reactor. The productivity of a gas agitated multiphase reactor, as shown in FIG. 3, also is affected by the diameter of the reactor. The greater the diameter of the reactor, the lower the productivity. Therefore it is an advantage of the present invention to be able to achieve in a large diameter reactor a high productivity similar to that obtained from a small diameter reactor by changing the flow hydrodynamics patterns in order to minimize liquid axial dispersion and obtain similar hydrodynamics profile of a multitude of small diameter reaction zones within the larger reactor.

As stated previously, the spirit of the present invention comprises configuring the internal structures of a multiphase reactor so that the flow upward through the reactor is altered to optimize backmixing and/or create a more controllable axial dispersion coefficient. In some embodiments, the structures are arranged in various patterns to create repeating zones. Each zone will have a local hydrodynamic similar to a small diameter column. By effectively separating the column multiple zones, the flow upward through the column is more evenly distributed leading to a reduction of backmixing at a given gas velocity.

As described herein the present invention contemplates one or more reaction regions within the reactor having one or more configurations of internal structures, wherein each reaction zone is in fluid communication with at least one adjacent reaction zone. The configurations are sometimes referred to as reaction zones created through non-uniform distribution of internal structures within a reaction vessel. This reference is intended to distinguish the general embodiments of the present invention over configurations of fully uniform-equally spaced internal structures. The embodiments described herein are intended to have varying degrees of non-uniformity ranging from, but not including, fully uniform-equally spaced configurations to completely random configurations. It is contemplated that one embodiment may comprise non-uniformity as a function of the cross-sectional area of the reaction vessel. Specifically, a preferred embodiment may comprise a completely non-uniform configuration at 5% to 20% of the total cross-sectional area of the reaction vessel. Stated differently, dividing the cross-sectional area of a reaction vessel into zones of 5% to 20% of the total area, the formed zones should not comprise identical configurations and/or area of internal structures.

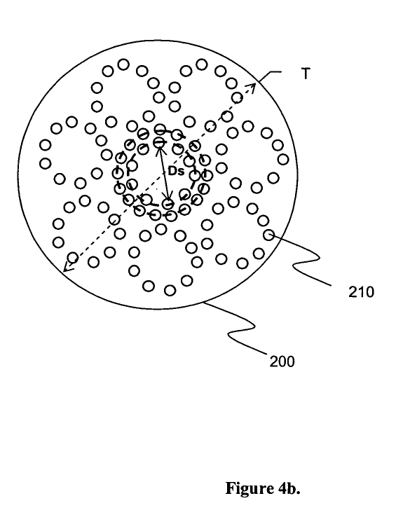

FIGS. 4-10 show examples of how the different regions may be represented within a multiphase reactor. As shown in FIG. 4a, a multiphase reactor has an outer wall 200 having an internal diameter Dr and one or more reaction regions 220, defined by various geometric configurations of internal structures 210. The internal structures 210 have a characteristic size, d, and the reaction regions have a characteristic size, Ds. The spacing between internal structures 210 in order to define the geometric patterns is characterized by Di, and is defined by the distance between centers of internal structures. Reactor internal diameter Dr is greater than Ds, which in turn is greater than d. Dr is preferably greater than about 0.6 meters (about 2 feet), more preferably greater than about 1.2 meters (about 4 feet), still more preferably greater than about 1.8 meters (about 6 feet). Ds is preferably from about 0.15 meters to about 0.6 meters (6-24 inches), more preferably from about 0.15 meters to about 0.5 meters (6-20 inches). The spacing, Di, between internal structures is preferably from about 1.1d and about 4d, more preferably from about 1.2d and about 3d. The diameter of internal tube, d, is preferably from about 2.5 cm to about 13 cm (about 1-5 inches), and more preferably from about 4 cm to about 10 cm (about 1.6-4 inches). The spacing between internal structures can be selected so that the liquid axial flow is reduced or minimized. It will be appreciated that more control of the liquid axial dispersion may be obtained the closer the structures are to each other. The spacing of the internal tubes may also be selected on their ability to break up large gas clouds.

FIGS. 4a and 4b show two cross sections of a multiphase reactor with a circular arrangement of the internal structures 210. As shown, the internal structures 210 are tubes that have been arranged into circular zones. FIG. 4a shows at the center of the reactor a circular zone defined for this example with internal structures. FIG. 4b on the other end shows at the center of the reactor another circular zone defined for this example with two concentric circular arrangements of internal structures. The concentric arrangement as shown in FIG. 4b is expected to further reduce the liquid axial dispersion within the small reaction region; however the reactor slurry is still in communication between the adjacent reaction region(s) and is permitted to move to and from the adjacent reaction region(s). In both embodiments with circular regions, it is preferable to have at least 2 distinct circular regions more preferably at least 4 circular regions.

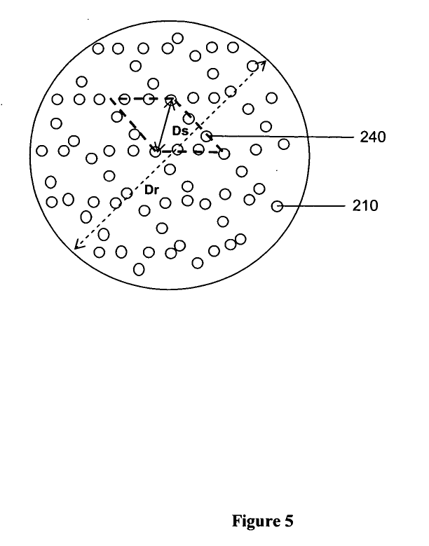

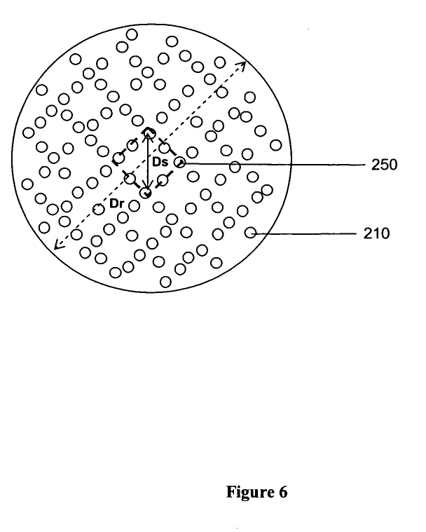

The patterned arrangements of internal structures may create a cross-sectional shape of the reaction zones selected from the group consisting of circular (FIG. 4b), concentric circular (FIG. 4b), diamond (FIG. 5), rectangular (FIG. 6), and any combination thereof; however it should be appreciated that the configurations shown in FIGS. 4-10 are not intended to be limiting or exhaustive of all possible configurations of the present invention. They are merely examples provided to illustrate of the spirit of the invention. In addition, the drawings are of cylindrical reactors and tubes, but any shape can be used without departing from the principles described herein. Thus, the scope of the present invention is not intended to be limited to any particular shape of reactor or internal structures.

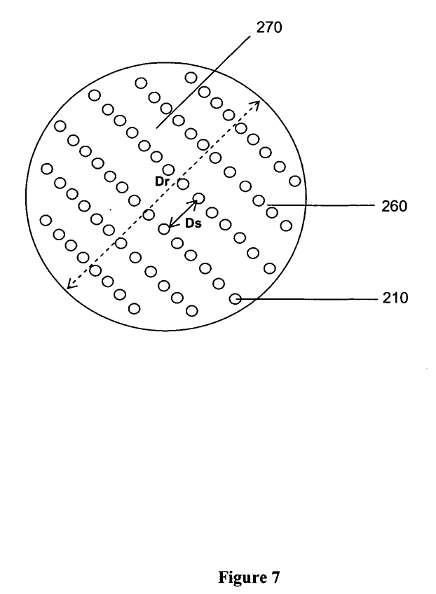

FIG. 5 shows a cross section of a multiphase reactor with repeating diamond shape arrangement 240 of the internal structures 210. FIG. 6 shows a cross section of a multiphase reactor with repeating rectangular shape arrangement 250 of the internal structures 210. FIG. 7 shows a cross section of a multiphase reactor with repeating parallel lines 260 of internal structures 210. The parallel lines of internal structures create parallel zones 270.

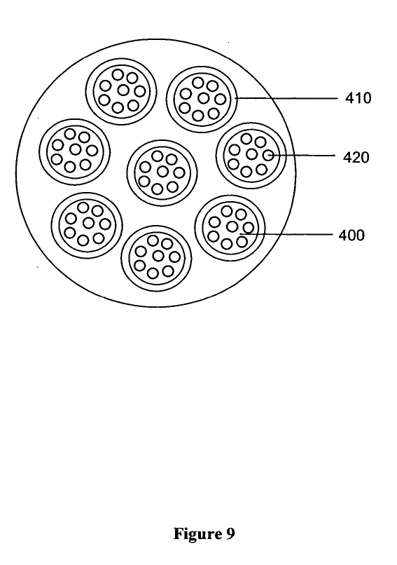

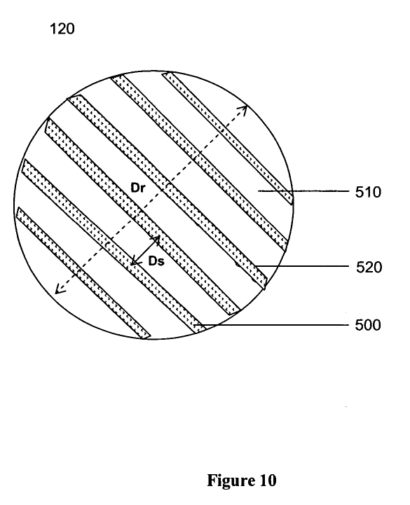

FIG. 8 shows a cross section of a multiphase reactor with internal heating or cooling tubes 320 and internal tubular structures 300 with walls 310 permeable to gas/liquid such that during operation the solid phase will be retained outside said walls 310. FIG. 9 shows a cross section of a multiphase reactor with internal heating or cooling tubes 420 and internal tubular structures 400 with walls 410 permeable to gas/liquid and with solid phase retained inside said walls 410. FIG. 10 shows a cross section of a multiphase reactor with repeating parallel internal structures 500 with walls 510 permeable to gas/liquid. The parallel internal structures create parallel zones 520. These 3 embodiments in FIGS. 8-10 have the similar hydrodynamic advantage of the multiple smaller reaction regions (in terms of total reactor inner diameter), and in addition, allow the liquid/gas to permeate through the walls of the internal structures thereby separating the products and reactants from the solid particles present in the slurry.

Another embodiment of the present invention comprises a Fischer-Tropsch multiphase reactor, preferably slurry bubble column reactor. Although Fischer-Tropsch reactors are expressly mentioned, the present invention is equally applicable to other types of multiphase reactors. Fischer-Tropsch reactors are expressly mentioned herein only as a preferred embodiment and for the sake of clarity and illustration. One skilled in the art will readily understand the applicability of the present invention towards other multiphase reactors. Thus, this specificity should not be interpreted as limiting but instead the present invention should be limited only by the claims as provided.

Nonetheless, in a preferred embodiment, the multiphase reactor will comprise a Fischer-Tropsch reactor. Any Fischer-Tropsch multiphase technology and/or methods known in the art will suffice. The feed gases charged to the process of the invention comprise hydrogen, or a hydrogen source, and carbon monoxide. H₂/CO mixtures suitable as a feedstock for conversion to hydrocarbons according to the process of this invention can be obtained from light hydrocarbons such as methane by means of steam reforming, autothermal reforming, partial oxidation, from coal by gasification, from biomass, or other processes known in the art. Preferably the hydrogen is provided by free hydrogen, although some Fischer-Tropsch catalysts have sufficient water gas shift activity to convert some water and carbon monoxide to carbon dioxide and hydrogen for use in the Fischer-Tropsch process. It is preferred that at least a portion of the feedstock comprising H₂ and CO is derived from a catalytic partial oxidation of light hydrocarbons, such as for example, those described in co-owned U.S. Pat. Nos. 6,402,989 and 6,409,940, and US published patent application 2002/0115730, all of which are hereby incorporated by reference. It is preferred that the molar ratio of hydrogen to carbon monoxide in the feed be greater than 0.5:1 (e.g., from about 0.67 to 2.5). Preferably, when cobalt, nickel, and/or ruthenium catalysts are used, the feed gas stream contains hydrogen and carbon monoxide in a molar ratio of about 1.8 to 2.3:1. Preferably, when iron catalysts are used the feed gas stream contains hydrogen and carbon monoxide in a molar ratio between about 1.4:1 and 2.2:1. The feed gas may also contain carbon dioxide. The feed gas stream should contain a low concentration of compounds or elements that have a deleterious effect on the catalyst, such as poisons. For example, the feed gas may need to be pretreated to ensure that it contains low concentrations of sulfur or nitrogen compounds such as hydrogen sulfide, hydrogen cyanide, ammonia and carbonyl sulfides.

The Fischer-Tropsch process is typically run in a continuous mode. In this mode, the gas hourly space velocity through the reaction zone typically may range from about 50 to about 10,000 hr⁻¹, preferably from about 300 hr⁻¹ to about 2,000 hr⁻¹. The gas hourly space velocity is defined as the volume of reactants per time per reaction zone volume. The volume of reactant gases is at standard conditions (standard pressure of 1 atm (101 kPa) and standard temperature of 0° C. (273.16 K)). The reaction zone volume is defined by the portion of the reaction vessel volume where reaction takes place and which is occupied by a gaseous phase comprising reactants, products and/or inerts; a liquid phase comprising liquid/wax products and/or other liquids; and a solid phase comprising catalyst. The reaction zone temperature is typically in the range from about 160° C. to about 300° C. Preferably, the reaction zone is operated at conversion promoting conditions at temperatures from about 190° C. to about 260° C. The reaction zone pressure is typically in the range of about 80 psia (552 kPa) to about 1000 psia (6895 kPa), more preferably from 80 psia (552 kPa) to about 600 psia (4137 kPa), and still more preferably, from about 140 psia (965 kPa) to about 500 psia (3447 kPa).

As the syngas feedstock is introduced into the Fischer-Tropsch reactor the gas bubbles up and through the slurry column. The Fischer-Tropsch reactor column should be configured according to the principles of the present invention. The gas generally serves to maintain some level of mixing as it transfers up the column. As the gas moves upward, it comes in contact with the catalyst material and the hydrocarbon synthesis reaction takes place. In addition, the gas will come in contact with the internal structures creating a more even distribution of smaller gas bubbles, which should enhance the productivity and conversion in the reactor. Products are formed including hydrocarbons and water. Water is a by-product of the Fischer-Tropsch reaction as shown in equation (1).

CO+2H₂→—(CH₂)—+H₂O (1)

Fischer-Tropsch catalysts are well known in the art and generally comprise a catalytically active metal, a promoter and a support structure. The most common catalytic metals are Group 8, 9 and 10 of the periodic table metals, such as cobalt, nickel, ruthenium, and iron or mixtures thereof. The preferred metals used in Fischer-Tropsch catalysts with respect to the present invention are cobalt, iron and/or ruthenium, however, this invention is not limited to these metals or the Fischer-Tropsch reaction. Other suitable catalytic metals include group 8, 9 and 10 metals. The promoters and support material are not critical to the present invention and may be comprised, if at all, by any composition known and used in the art. The preferred support compositions are alumina, silica, titania, zirconia or mixtures thereof.

As stated above, the feedstock for a Fischer-Tropsch reaction is syngas, i.e., gas comprised mainly of hydrogen and carbon monoxide. Typically, syngas is produced in a syngas reactor in connection with producing Fischer-Tropsch products. According to the present invention, a syngas reactor can comprise any of the synthesis gas technology and/or methods known in the art. Similarly, the oxygen-containing gas may come from a variety of sources and will be somewhat dependent upon the nature of the reaction being used. For example, a partial oxidation reaction requires diatomic oxygen as a feedstock while steam reforming requires only steam. According to the preferred embodiment of the present invention, partial oxidation is assumed for at least part of the syngas production reaction.

The synthesis gas feedstocks are generally preheated, mixed and passed over or through the catalyst beds. As the mixed feedstocks contact the catalyst the synthesis reactions take place. The synthesis gas product contains primarily hydrogen and carbon monoxide, however, many other minor components may be present including steam, nitrogen, carbon dioxide, ammonia, hydrogen cyanide, etc., as well as unreacted feedstock, such as methane and/or oxygen. The synthesis gas product, i.e., syngas, is then ready to be used, treated, or directed to its intended purpose. For example, in the instant case some or all of the syngas will be used as a feedstock for the Fischer-Tropsch process.

While preferred embodiments of this invention have been shown and described, modification thereof can be made by one skilled in the art without departing from the spirit or teaching of this invention. The embodiments described herein are exemplary only and are not limiting. Many variations and modifications of the processes are possible and are within the scope of this invention. For example, there are numerous configurations of internal structures that can help reduce backmixing without departing from the spirit of the present invention. Also, although the internal structures are referred to in the plural, it is within the scope of the present invention that all of the internal structures are a single unit. For example, if the structures are part of cooling coils, the coil may be a continuous set of vertical tubes connected by a connection means such as alternating u-ends with an inlet and outlet tube or a manifold system that is in communication with the vertical tubes. Likewise, the structures may be a several sets of coils with multiple vertical components. In yet another example, it is contemplated that the internal structures may be of varying size, in which case the references to diameter d of such tubes is intended as the average diameter of the tubes in the region in which they are located. Likewise, the references to distances between tubes and rows in this situation is also intended as an average of those respective values for the region in which they are located.

Further, it should be appreciated that the term discrete when referring to the reaction flow zones is not intended to mean a completely individualized reaction chamber, but rather a region that can be identified as behaving like another region within the reactor. It should also be contemplated that the internal structures may be tubes or rods of various cross-sectional shapes (circular, trilobe, oval, rectangular or square, or irregular-shaped for example), in which case the references to diameter d of such structures would be their maximum width. It should also be understood that the drawings are meant to be exemplary and not limiting. Any non-uniform configuration (as defined herein) is suitable. Although not limiting, in preferred embodiments the internal structures occupy about 10% to 25%, preferably about 15% to 25%, more preferably about 15% to 20% of the cross-sectional area of the reaction vessel. Those of ordinary skill in the art will appreciate that many other variations are possible and within the spirit and scope of the present invention. Accordingly, the scope of protection is not limited to the embodiments described herein, but is only limited by the claims that follow, the scope of which shall include all equivalents of the subject matter of the claims. In addition, unless order is explicitly recited, the recitation of steps in a claim is not intended to require that the steps be performed in any particular order, or that any step must be completed before the beginning of another step.

What is claimed is:

1. A process for producing liquid hydrocarbons from synthesis gas comprising:
contacting a gas stream comprising hydrogen and carbon monoxide in a multiphase reactor comprising a reaction vessel having an internal diameter (Dr) of greater than or equal to 0.6 m and a plurality of internal structures arranged within said reaction vessel under operating conditions effective to convert at least a portion of the gas streams to liquid hydrocarbon synthesis products, wherein the internal structures are arranged such that they create a plurality of reaction zones within the reaction vessel, wherein each reaction zone is in fluid communication with at least one adjacent reaction zone, and wherein the plurality of internal structures is configured such that each of the reaction zones has a characteristic size Ds that is less than the reaction vessel internal diameter Dr.

2. The process according to claim 1 wherein Dr is in the range of about 0.6 m to about 10 m.

3. The process according to claim 1 wherein Dr is equal to or greater than 10 m.

4. The process according to claim 1 wherein the plurality of reaction zones is created by patterned arrangements of the internal structures.

5. The process according to claim 4 wherein the patterned arrangements comprise create a cross-sectional shape of the reaction zones selected from the group consisting of circular, rectangular, diamond, concentric circular, and any combination thereof.

6. The process according to claim 1 wherein the structures are arranged in various patterns to create repeating reaction zones.

7. The process according to claim 1 wherein Ds is between about 0.15 meter and about 0.6 meter.

8. The process according to claim 1 wherein each of the plurality of internal structures has a characteristic size d, and wherein d is smaller than Ds.

9. The process according to claim 1 wherein each of the plurality of internal structures has a characteristic size d, and the spacing Di between centers of adjacent internal structures is between about 1.1d and about 4d.

10. The process according to claim 9 wherein d is from about 2.5 cm to about 13 cm (about 1-5 inches).

11. The process according to claim 1 wherein the reaction vessel has a height to diameter ratio between about 0.5 and about 20.

12. The process according to claim 1 wherein each of the reaction zones has a height to diameter ratio between about 7 and about 180.

13. The process according to claim 1 wherein the internal structures include heating or cooling tubes.

14. The process according to claim 1 wherein the internal structures are parallel so as to create parallel reaction zones.

15. The process according to claim 1 wherein the synthesis products comprise C₅+hydrocarbons.

16. The process according to claim 1 wherein the internal structures comprise an area of about 10% to about 25% of the cross-sectional area of the reaction vessel.

17. The process according to claim 1 wherein the internal structures comprise a non-uniform configuration.

18. The process according to claim 17 wherein the internal structures comprise a completely non-uniform configuration at 5% to 20% of the total area of the reaction vessel.

19. A gas-agitated multiphase reactor with a low degree of backmixing suitable for hydrocarbon synthesis, comprising:
a reaction vessel characterized by an internal diameter Dr of greater than or equal to 0.6 m;
a liquid disposed inside the reaction vessel;
a gas distributor disposed near the bottom of the reaction vessel, said gas distributor being suitable for dispersing a gas phase through the liquid and creating a gas flow and a fluid flow; and
a plurality of internal structures disposed within said reaction vessel,
wherein the plurality of internal structures is arranged so as to create a plurality of reaction zones within the reaction vessel,
wherein each reaction zone is in fluid communication with at least one adjacent reaction zone, and
wherein the plurality of internal structures is configured such that each of said reaction zones has a characteristic size Ds that is less than the reaction vessel internal diameter Dr.

20. The reactor according to claim 19 wherein the plurality of reaction zones is created by patterned arrangements of internal structures.

21. The reactor according to claim 20 wherein the patterned arrangements comprise create a cross-sectional shape of the reaction zones selected from the group consisting of circular, rectangular, diamond, concentric circular, and any combination thereof.

22. The reactor according to claim 20 wherein the structures are arranged in various patterns to create repeating zones.

23. The reactor according to claim 19 wherein Dr is in the range of 0.6 m to 10 m.

24. The reactor according to claim 23 wherein Dr is greater than or equal to about 1.2 meters.

25. The reactor according to claim 24 wherein Dr is greater than or equal to about 1.8 meters.

26. The reactor according to claim 19 wherein Dr is greater than or equal to 10 m.

27. The reactor according to claim 19 wherein Ds is between about 0.15 meter and about 0.6 meter.

28. The reactor according to claim 19 wherein Ds is between about 0.15 meter and about 0.5 meter.

29. The reactor according to claim 19 wherein the reaction vessel has a height to diameter ratio between about 0.5 and about 20.

30. The reactor according to claim 19 wherein each of the reaction zones has a height to diameter ratio between about 7 and about 180.

31. The reactor according to claim 19 wherein each of the plurality of internal structures has a characteristic size d, and wherein d is smaller than Ds.

32. The reactor according to claim 19 wherein each of the plurality of internal structures has a characteristic size d, and the spacing Di between centers of adjacent internal structures is between about 1.1d and about 4d.

33. The reactor according to claim 19 wherein Di is between about 1.2d and about 3d.

34. The reactor according to claim 19 wherein Di is between about 1.2d and about 3d.

35. The reactor according to claim 19 wherein d is from about 2.5 cm to about 13 cm (about 1-5 inches).

36. The reactor according to claim 19 wherein d is from about 4 cm to about 10 cm (about 1.6-4 inches).

37. The reactor according to claim 19 wherein the plurality of internal structures comprises components having walls that are permeable to gas or liquid.

38. The reactor according to claim 19 wherein the reaction vessel further includes a solid phase and said solid phase is retained outside said walls during operation.

39. The reactor according to claim 19 wherein the reaction vessel further includes a solid phase and said solid phase is retained inside said walls during operation.

40. The reactor according to claim 19 wherein the internal structures are parallel so as to create repeating parallel reaction zones.

41. The reactor according to claim 19 wherein the internal structures includes tubes or rods.

42. The reactor according to claim 19 wherein the internal structures comprise components having cross-sectional shapes selected from the group consisting of circular, trilobe, oval, rectangular, square, and irregular shapes.

43. The reactor according to claim 19 wherein the internal structures include heating or cooling tubes.

44. The reactor according to claim 19 wherein the multiphase reactor further comprises one or more tubular structures wherein the tubular structures are permeable to gas and liquid.

45. The reactor according to 19 wherein the gas-agitated multiphase reactor is a hydrocarbon synthesis reactor.

46. The reactor according to 19 wherein the gas-agitated multiphase reactor is a slurry bubble column.

47. A method for reducing backmixing in a large scale gas-agitated multiphase reactor comprising:
providing a reactor vessel having a bottom and a plurality of internal structures arranged within said reaction vessel and an internal diameter Dr greater than or equal to about 0.6 m, wherein said reaction vessel contains a liquid, wherein the internal structures are arranged so as to create a plurality of reaction zones within the reaction vessel, and wherein each of said reaction zones has a characteristic size Ds that is less than the reaction vessel internal diameter Dr; and passing a gas phase from the bottom of the reactor vessel through said liquid into the plurality of reaction zones so as to create a liquid flow in each of the reaction flow zones; wherein the liquid flow in each of the reaction flow zones has a liquid axial dispersion coefficient lower than that of a liquid flow in the reaction vessel without internal structures.

48. A reactor comprising:
a large diameter reaction vessel capable of having liquid contained therein;
a means for introducing gas into the reaction vessel; and
a means for reducing the liquid axial dispersion coefficient and backmixing within the reaction vessel.

49. The reactor of claim 48 wherein the means for reducing comprises a non-uniform distribution of internal structures.

50. The reactor of claim 48 wherein the reaction vessel has a diameter of greater than or equal to 0.6 m.
